(19) United States
(12) Patent Application Publication
(10) Pub. No.: US 2007/0190595 A1
(43) Pub. Date: Aug. 16, 2007

(54) Process for obtaining zeaxanthin from algae

(76) Inventors: Albrecht Weiss, Langenfeld (DE); Wilhelm Johannisbauer, Erkrath (DE); Bernhard Gutsche, Hilden (DE); Baldomero F. Cordero, Bormujos Sevilla (ES); Lucia Martin, Sevilla (ES); Herminia Rodriguez, Sevilla (ES); M. Angeles Vargas, Dos-Hermanes Sevilla (ES); Irina Obraztsova, Sevilla (ES)

Correspondence Address: COGNIS CORPORATION;PATENT DEPARTMENT, 300 BROOKSIDE AVENUE, AMBLER, PA, 19002, US

(21) Appl. No.: 11/650,264

(22) Filed: Jan. 5, 2007

(30) Foreign Application Priority Data
Jan. 5, 2006 (EP) 06000158.3

(51) Int. Cl. C12P 1/00

(52) U.S. Cl. 435/41

(57) ABSTRACT

A process of obtaining zeaxanthin in a high yield from blue-green algae is de-scribed. Zeaxanthin and zeaxanthin-enriched products obtained by the process, which are suitable for use as dietary supplements and/or food additives, are also described.

### CROSS-REFERENCE TO RELATED APPLICATIONS

This application claims priority under 35 USC Section 119 of European patent application, EP06000158 filed Jan. 5, 2006, which is incorporated herein in its entirety.

### FIELD OF THE INVENTION

The present invention relates to a process for obtaining zeaxanthin from blue-green algae for the production of zeaxanthin and zeaxanthin-enriched products.

### BACKGROUND OF THE INVENTION

Zeaxanthin, together with lutein, is an essential component of the macular pigment in the retina of the eye. A low level of intake of this particular carotenoid increases the risk of age-related macular degeneration (AMD) and cataracts, which are the leading causes of visual impairment and acquired blindness, and are key quality of life issues among millions of ageing people. One of the first large-scale studies on carotenoids is the Eye Disease Case Control Study, in which diet was compared to the risk of developing AMD. Results found a significantly lower risk of developing the eye disease in people showing high amounts of lutein and zeaxanthin in their blood. Also, the people who followed a diet with the highest amounts of lutein and zeaxanthin developed a significantly lower risk of AMD than those whose diet contained the least amount (as low as 1.2 mg per day). Dietary studies confirmed the association between frequent consumption of spinach or collard greens, which are particular good sources of lutein and zeaxanthin and of lower AMD risk. Similar results were found in a recent analysis of a US dietary study called the Third National Health and Nutrition Examination Survey or NHANES III. This analysis also showed that consuming lutein and zeaxanthin was associated with a reduced risk of developing AMD. While lutein is relatively abundant in the food we eat, zeaxanthin is not that easily obtained through a well-balanced diet, and thus should be added as a food additive.

Zeaxanthin (3,3′-dihydroxy-β-carotene) represents an oxygenated carotenoid or xanthophyll. The conjugated double bond system determines its color (yellow-red) and is responsible for its biological activity.

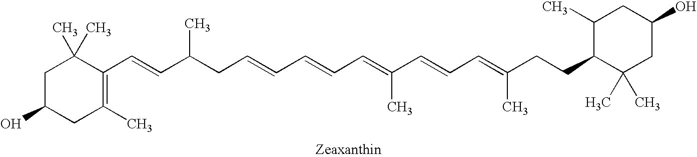

The physiological properties of zeaxanthin, and particularly its function as an antioxidant, are due to its potential to inactivate singlet oxygen and to quench active radicals. Up to today, zeaxanthin is mainly produced synthetically since the content of this carotenoid in natural sources is considered to be rather low for any industrial production under economic conditions. For example, zeaxanthin is present in some bacteria (e.g. Flavobacterium, Paracoccus, Halobacterium, Xanthobacter or Erwinia) and higher plants, where they play a role in the so-called Xanthophyll Cycle. In the case of Flavobacterium, the volumetric content in zeaxanthin is 10.6 mg/l after a six-fold improvement by using different intermediates of the citric acid cycle. With regard to the micro-alga Neo-spongiococcum excentricum, the content of zeaxanthin of the wild type is 0.35 mg/g dry weight. In a constitutive mutant of Dunaliella salina the content of zeaxanthin has been found to be about 6 mg/g dry weight (see Jin et al., Biotechnol. Bioeng. 81:115-124 (2002)).

The complex problem underlying the present invention has therefore been to develop a process which obtains zeaxanthin from natural sources in higher yields compared to the state of the art, more particularly from algae showing simultaneously:
a specific growth rate μ at medium light conditions of at least 0.07 h⁻¹ (measured under phototropic conditions using inorganic media);
a zeaxanthin/lutein ratio of more than 10;
a factor obtained by multiplication of growth rate and zeaxanthin volumetric productivity of at least 0.01 mg⁻¹h⁻²;
a chlorophyll A l zeaxanthin ratio of less than 10, and
a volumetric zeaxanthin productivity of at least 0.04 mg⁻¹h⁻¹.

In addition, the starting material should be easy to cultivate and to harvest, so that it is possible to carry out the reaction in a photo-bioreactor. Finally, the sources should be free of any harmful toxins or to be cultivated under such non-toxin-producing conditions to avoid harmful toxin formation, and be resistant to contamination.

### BRIEF DESCRIPTION OF THE INVENTION

The present invention is directed to a process for obtaining natural antioxidants, which provides for:
(a) cultivating a blue-green algae selected from the group consisting of Anacystis nidulans (L-1402-1), Synechococcus sp. (PCC 7492) and Synechocystis sp. (PCC 6803) under cultivating conditions suitable for producing zeaxanthin;
(b) harvesting the cultivated algae containing zeaxanthin; and
(c) obtaining the zeaxanthin from the harvested algae containing zeaxanthin.

Surprisingly, it had been found that blue-green algae (cyanobacteria) described above, particularly Anacystis nidulans (L-1402-1), produce the desired zeaxanthin in a significantly higher amount compared to any other known micro-organism or plant reported in the state of the art.

The blue-green algae which are useful in the practice of the invention, and in particular Anacystis, also show a number of additional advantages compared to other algae: they exhibit a quick growth (doubling time from 3.6 to 5.3 h); they do not clump, and are easy to handle, so that they are suitable for cultivation, especially in tubular photo-bioreactors. They grow auto- or mixotrophically in a very simple and cheap mineral medium, and are resistant with respect to contamination.

### DETAILED DESCRIPTION OF THE INVENTION

Other than in the operating examples, all numbers expressing quantities of ingredients or reaction conditions are to be understood as being modified in all instances by the term “about.”

Blue-green Algae

Among the three strains cited above, Anacystis nidulans (L-1402-1) is preferred, since it does not only exhibit the highest productivity with respect to zeaxanthin, but also shows the best resistance against pollutants, and exhibits the fastest growth rate. Nevertheless, it shall be understood that the present invention is not limited to these wild forms, but also encompasses any mutants of these strains including forms obtained by genetic modification or engineering.

Cultivation Conditions

Many culture conditions and culture media are known for small-scale stock cultures and large-scale cultures of algae cells. However, for the purposes of the present invention, it was found that the optimal culture conditions and media are the relatively simple culture conditions and media described herein below; these therefore being the preferred ones in accordance with the invention. For example, temperature is a factor for the growth of algae. It has been found that very favorable conditions are achieved at between about 20 and 40° C. with a temperature of about 30° C. being preferred.

In a further preferred embodiment of the invention, the algae are grown mixotrophically (with additional nutrients to enhance cell growth), more particularly, Arnon medium has been found to support the growth in an optimal manner, preferably in combination with nitrates. The growth rate can also be increased by the addition of salts of acetic acid, in particular sodium acetate, in amounts of about 0.1 to 20 mM, preferably about 15 mM.

Irradiation of the cells during cultivation can also be carried out. As far as screening and pilot plant tests are concerned, mercury halide lamps are typically used, and cultivation is carried out under a light irradiation of about 500 to 1,500, and preferably about 1,200 μEm⁻²s⁻¹. For production, natural light conditions are preferred.

In a further embodiment of the present invention, the cells are cultivated in a photo-bioreactor, preferably a tubular or a panel photo-bioreactor, having the advantage of a very large surface area with respect to its volume for optimal large-scale production of algae cells in their growth phase. Usually, such bioreactors have a volume in the range from 100 to 35,000 liters, depending on the scale of production that is desired. Components contain commercially available tubes made of PVC, acrylic (Plexiglas™), polycarbonate or glass, having a diameter of about 3 to 5 cm. In operation, the culture cells are circulated through the device in tap water to which CO₂ is added, using a pump. The green cultures from the liquid stock cultures (or inoccula) are inoculated into the photo-bioreactor, in which the algae are sparged with a CO₂ containing gas, such as a mixture of CO₂ in air or CO₂ as the main gas component. Sparging and pumping also aid in preventing the clumping of the cells. For production scale in a tubular photo-bioreactor, 90 to 100% pure CO₂ can be utilized; inexpensive CO₂ from industrial plants (e.g. from a quick lime process) is especially suitable.

The initial cell concentration in the photo-bioreactor during process cultivation is preferably adjusted from about 0.1 to 0.3×10⁶ cells/ml at cell concentrations (dry mass) between 0.1-8 g dry biomass/l, by dilution with fresh modified cultivation medium. The light intensity is usually kept in the range of between 500 and 1,500 μEm⁻²s⁻¹ as provided by mercury halide lamps or as natural light. It has been found advantageous to maintain the temperature in the photo-bioreactor in the range between 25 and 28° C. By using a glass house as an indoor cultivation room, the temperature can be maintained below 32° C., as a non-limiting example, in central and northern Europe in summer time. In addition, the tubular photo-bioreactor and the indoor placement have the advantage of clean, long term and controlled operation. If the photo-bioreactor is made of glass tubes instead of plastic, extensive maintenance or revamping in order to operate for a long time is not necessary. Further, the culture conditions are extremely inexpensive, as the cells are grown in a cheap mineral medium of tap water with the addition of carbon dioxide as, essentially, the major nutrient source.

Harvesting and Recovery of Zeaxanthin

In order to obtain zeaxanthin or a zeaxanthin-enriched product from cultivated algae cells, many procedures have been described, for example, the procedures in WO 89/006910. While these procedures may be employed in accordance with the present invention, the preferred procedure is that of centrifugation, or sedimentation or filtration under vacuum to concentrate the cells, and drying of the concentrated cells. The dried cell mass is then preferably stored at low temperatures (e.g., −20° C. or even lower) under oxygen-free conditions, e.g., by vacuum packing or, preferably, by introduction into plastic containers maintained under nitrogen (N₂) to remove the oxygen.

In a further embodiment of the invention, the process comprises the following additional steps:
(i) harvesting the cells cultivated in step (b) by collecting said cells to form a concentrated suspension,
(ii) optionally adding antioxidants and emulsifiers to said suspension, and
(iii) disrupting the collected cells and drying them to obtain a zeaxanthin or a zeaxanthin-enriched product.

Generally, the collection and concentration of the cells is carried out by centrifugation, or sedimentation or filtration under vacuum, and the drying of said cells is done by lyophilization, combined with drying and grinding (air-vortex-mill), or spray drying.

More particularly, while each cultivation cycle optimally lasts about four to six days, the following harvesting procedure is performed based on the fact that algae cells readily sediment once collected from the photo-bioreactor. Thus, the cell biomass from the photo-bioreactor is collected into a standard large volume funnel, e.g. an Imhoff funnel, and allowed to stand for a few hours (about 3-5 hours) to facilitate sedimentation of the cells. It was found that approximately 30% b.w. of the total volume of biomass from the bioreactor represented the cell sediment while the remaining approx. 70% b.w. of the total collected volume represented the tap water used in the cultivation. This tap water can thus be easily collected and used for a new bioreactor inoculation and cultivation procedure (i.e., the originally used tap water is almost completely recyclable). The above precipitated and concentrated cell culture is then collected from the funnel and subjected to centrifugation or vacuum filtration to further concentrate the cells. Routinely, a biomass yield of about 40% b.w. solids is obtained following the centrifugation step, or about 30% b.w. solids following vacuum filtration. Here, too, the approximately 60% b.w. of the total volume subjected to centrifugation, or approximately 70% b.w. of the total volume subjected to vacuum filtration, being the supernatant volume, could also be collected and used for another round of the cultivation procedure, this supernatant being primarily the original tap water used in the procedure.

The concentrated cell slurry obtained from the above centrifugation step is then homogenized and stabilized by adding antioxidants and then dried, preferably by lyophilization, although spray drying also can be utilized. Such antioxidants are selected from the group consisting of ethoxyquin, butylated hydroxyanisole, butylated hydroxytoluene (BHT), tocopherols, di-tert-butyl-paracresol and propyl gallate. The preferred antioxidant is a natural tocopherol product containing 30% b.w. of alpha tocopherol. Usually, the amount of antioxidant added in the grinding procedure will range from about 0.05 to 5% (w/w) of the amount of dry powder. The powders are packed into a plastic bag pre-filled with nitrogen gas to remove oxygen (which causes pigment oxidation, i.e. degradation of the active) and are then stored at −20° C. prior, e.g., to processing to prepare a food additive or supplement.

The final stage of producing a zeaxanthin or a zeaxanthin-enriched product in the form of small particles easily digested by humans or animals may also be carried out in a number of ways as previously described in the art. Thus, zeaxanthin and other algae components are processed to assure a high bioavailability. The preferred procedure involves the use of a standard ball mill, in which the biomass slurry is disintegrated as a suspension in water in the presence of any suitable antioxidant to prevent oxidation of the zeaxanthin. After drying, this yields a powder-like product of small particle size.

The powder thus obtained may then be utilized directly or in admixture with other ingredients as an additive to fish meal for coloration or in food applications like dietary supplements. In another process, the zeaxanthin can be concentrated by an extraction process including extraction with supercritical solvents, which zeaxanthin is suitable for use in the formulation of food supplements or pharmaceutical products.

According to the teaching of the present invention, certain blue-green algae have been found to exhibit a surprisingly high productivity for the production of zeaxanthin. Another object of the present invention is therefore directed to the use of blue green algae selected from the group of strains consisting of Anacytis nidulans (L-1402-1), Synechococcus sp. (PCC 7492) and Synechocystis sp. (PCC 6803), either in their wild forms or in the form of any mutant, including those forms obtained by genetic modification or engineering, for the production of zeaxanthin or zeaxanthin-enriched products.

Another embodiment of the invention then pertains to the use of zeaxanthin, including the zeaxanthin-enriched biomass as directly obtained from cultivation, obtained herein as a food or feed additive or pharmaceutical product.

The following examples are illustrative of the invention and should not be construed in any manner whatsoever as limiting the scope of the invention.

### EXAMPLES

### Zeaxanthin Analysis

For the analysis of zeaxanthin, the pigments were extracted with methanol at 70° C., centrifuged, the supernatant evaporated under N₂ at 30° C.; and the pellet resuspended in acetone, centrifuged and analyzed by HPLC using a Waters Spherisorb S5 ODSI 4.6×250 mm cartridge column. The pigments were detected by using a photodiode-array detector.

### Cell Growth Conditions

Stock cultures of micro-algae were grown photoautotrophically in batch culture, in 100 ml of either Arnon culture medium (Arnon medium, modified to contain 4 mM K₂HPO₄ and 20 mM NaNO₃) or BG11c medium (Blue-green medium, www.ccap.ac.uk) in conical flasks of 200 ml capacity and illuminated with fluorescent lamps at 92 μE m⁻² s⁻¹. Culture temperature was 30° C. The screening of the strains for the production of zeaxanthin was performed under the following conditions:
Photoautotrophic batch cultures in Roux Flasks (750 ml) were started with cells at the exponential phase from the stock cultures at 0.7-0.8 mg (chlorophyll) l⁻¹
Irradiance: Continuous, 920-1610 μEm⁻²s⁻¹ (mercury halide lamps)
Temperature: 30° C. Bubbling: The cultures were bubbled with air supplemented with 1% (V/V) CO₂.
Culture medium: The same as in the case of stock culture.

Several parameters were analyzed in the different micro-algae, as cell density, dry weight, carotenoids and chlorophylls, pH, cell morphology and the specific growth rate (μ). Growth was followed by determining the chlorophyll A content (mgl⁻¹), or by dry weight (gl⁻¹). For dry weight determination, 5 ml aliquots were filtered through previously weighted 0.45 μm diameter Millipore filters, washed twice with distilled water and dried at 80° C. for 24 h. Chlorophyll A was determined spectrophotometrically in methanol extracts employing the extinction coefficient given by Mackinney. For mixotrophic growth, cultures were supplemented with 15 to 20 mM sodium acetate (experimental) and for nitrate optimization; the media were supplemented with 10 to 40 mM sodium nitrate. For the optimization of irradiance, light irradiances from 92 μEm⁻²s⁻¹ to 1610 μEm⁻²s⁻¹ were assayed. Temperature ranged from 22° C. to 40° C. in the experiment for temperature optimization. The medium was optimized for Anacystis nidulans L-1402; the following media were used: BG11c, Arnon medium supplemented with 20 mM nitrate, and BP medium (Jaworski's medium, catalogue of strains CCAP).

### Selection of Blue-green Micro-algae Strains

The following Tables 1 and 2 show a comparison of different blue-green micro-algae strains with respect to the productivity for zeaxanthin (P) and the factor “specific growth rate” multiplied with “zeaxanthin productivity”. As outlined above, the algae were cultivated in BG11c Medium at 30° C., the irradiance was 350 Wm⁻² (1610 μEm⁻²s⁻¹). Only three of them showed a high content in zeaxanthin, both at the exponential phase of growth and at the end of the culture: Anacystis nidulans L-1402-1, Synechococcus sp. PCC 7942 and Synechocystis sp. PCC 6803. The three strains also exhibited the highest specific growth rates. Therefore, these strains presented the highest zeaxanthin productivities (from 3.0 to 11.2 mg l⁻¹ day⁻¹).

TABLE 1
Productivity of blue-green micro-algae for zeaxanthin

| | μ | Chl A | DW (max) | Zea (max) | P (Zea) |
|---|---|---|---|---|---|
| Strains/Results | [h⁻¹] | [mgl⁻¹] | [gl⁻¹] | [mgl⁻¹] | [mgl⁻¹h⁻¹] |
| Anabaena | 0.05 | 13.26 | 1.06 | 0.06 | 0.00 |
| sp. PCC 7120 | | | | | |
| Anacystis nidulans | 0.16 | 23.30 | | 11.20 | 0.33 |
| L-1402-1 | | | | | |
| Chlorogloeopsis | 0.02 | 12.80 | 3.90 | 1.10 | 0.01 |
| sp. PCC 6912 | | | | | |
| Dermocarpa | 0.03 | 14.00 | 2.80 | 1.60 | 0.01 |
| sp. PCC 7437 | | | | | |
| Nostoc caquena | 0.02 | 2.90 | 6.80 | 0.20 | |
| Nodularia chucula | 0.05 | 32.20 | 2.50 | 0.70 | 0.02 |
| Nostoc llaita | 0.02 | 56.60 | 11.20 | 0.70 | 0.00 |
| Nostoc sp. | 0.03 | 23.30 | 2.80 | 0.30 | 0.00 |
| PCC 9201 | | | | | |
| Nostoc paludosum | 0.06 | 38.10 | 4.40 | 0.01 | |
| PCC 9206 | | | | | |
| Synechococcus | 0.08 | 9.60 | | 4.70 | 0.12 |
| sp. PCC 7492 | | | | | |
| Synechocystis | 0.08 | 22.40 | 1.50 | 3.00 | 0.20 |
| sp. PCC 6803 | | | | | |

Chl A = chlorophyll A;
Zea = zeaxanthin;
Lut = lutein;
DW = dry weight,
P = productivity

TABLE 2
Specific Growth Rate * Productivity of
blue-green micro-algae for zeaxanthin

| | Chl A | Zea (max) | Zea (max) | SPGR * |
|---|---|---|---|---|
| Strains/Results | Z̅e̅a̅ (̅m̅a̅x̅)̅ | Lut (max) | D̅W̅ (̅m̅a̅x̅)̅ | P (Zea) |
| Anabaena sp. PCC 7120 | 221.00 | 1.20 | 0.06 | 0.000 |
| Anacystis nidulans | 2.08 | 14.00 | | 0.053 |
| L-1402-1 | | | | |
| Chlorogloeopsis | 11.64 | 1.22 | 0.28 | 0.000 |
| sp. PCC 6912 | | | | |
| Dermocarpa sp. PCC 7437 | 8;.75 | 2.00 | 0.57 | 0.000 |
| Nostoc caquena | 14.50 | | 0.03 | 0.000 |
| Nodularia chucula | 46.00 | 3.50 | 0.28 | 0.001 |
| Nostoc llaita | 83.71 | 0.47 | 0.06 | 0.000 |
| Nostoc sp. PCC 9201 | 77.67 | 0.14 | 0.11 | 0.000 |
| Nostoc paludosum | 3810.00 | | 0.000 | 0.000 |
| PCC 9206 | | | | |
| Synechococcus | 2.04 | 15.16 | | 0.010 |
| sp. PCC 7492 | | | | |
| Synechocystis | 7.47 | 2.50 | 2.00 | 0.016 |
| sp. PCC 6803 | | | | |

Chl A = chlorophyll A;
Zea = zeaxanthin;
Lut = lutein;
DW = dry weight,
SGR = Specific Growth Rate

### Kinetics of the Accumulation of Carotenoids

Table 3 shows the kinetics of accumulation of different carotenoids in Anacystis nidulans L1402-1, which was selected for further testing due to its particular high productivity of zeaxanthin. Zeaxanthin was the major carotenoid, but other carotenoids were present either in very low amounts or not detected at all. Zeaxanthin in the cultures increased with time, changing from 1.9 at the exponential phase to 11.2 mg l⁻¹ in the stationary phase. Total carotenoids followed the same trend, increasing from 2.5 to 14.7 mg l⁻¹ from the exponential to the stationary phase of culture.

### Effect of Growth Medium

In the following Tables 4, 5 and 6, optimized parameters with respect to culture medium, irradiance and acetate concentration for Anacystis nidulans are given. The highest cell growth, measured as chlorophyll content in the cultures, was obtained by using Arnon on medium supplemented with 20 mM of nitrate, whereas the lowest growth was observed served with BG11c medium. In particular, it was observed that the specific growth rate was higher in BG11c than in Arnon and BP medium, however, the highest zeaxanthin content was found in Arnon medium. Therefore, the highest zeaxanthin production occurred when cells were grown with Arnon medium, supplemented with 20 mM nitrate.

TABLE 3
Kinetics of the accumulation of carotenoids [mgl-1]

| | Exponential | | Deceleration | | Stationary | |
|---|---|---|---|---|---|---|
| Time [h] | phase | | phase | | phase | |
| Carotenoids | 12.5 | 18.5 | 24.5 | 38.0 | 94.5 | 170 |
| Violaxanthin | — | — | — | — | — | — |
| Astaxanthin | — | — | — | — | — | — |
| Anteraxanthin | 0.04 | 0.04 | 0.08 | 0.09 | 0.07 | 0.07 |
| Lutein | 0.06 | 0.07 | 0.10 | 0.14 | 0.61 | 0.82 |
| Zeaxanthin | 1.90 | 2.80 | 3.50 | 4.80 | 8.50 | 11.20 |
| Canthaxanthin | — | — | — | — | — | — |
| β-Cryptoxanthin | 0.14 | 0.07 | 0.11 | 0.16 | 0.23 | 0.30 |
| Lycopene | — | — | — | — | — | — |
| α-Carotene | 0.26 | 0.54 | 0.90 | 1.30 | 1.80 | 1.60 |
| β-Carotene | 0.07 | 0.12 | 0.22 | 0.43 | 0.80 | 0.74 |
| Total Carotenoids | 2.5 | 3.6 | 4.9 | 6.9 | 12.0 | 14.7 |

TABLE 4
Effect of growth medium

| | | Zea | | Zea | | |
|---|---|---|---|---|---|---|
| | μ | (max-E)* | Chl (max) | (max) | Zea Production | |
| Medium | [h⁻¹] | [mgl⁻¹] | [mgl⁻¹] | [mgl⁻¹] | mgl⁻¹h⁻¹ | mgl⁻¹d⁻¹ |
| BG11c | 0.19 | 2.3 | 12.0 | 6.5 | 0.44 | 10.6 |
| Arnon | 0.13 | 4.8 | 67.9 | 16.2 | 0.62 | 15.0 |
| BP | 0.12 | 4.8 | 25.9 | 8.7 | 0.58 | 13.9 |

*Maximal zeaxanthin content in exponential phase

In addition, it was observed that in the kinetics of zeaxanthin accumulation by Anacystis nidulans until the end of exponential phase, the accumulation of zeaxanthin in BP and Arnon medium was similar and higher than in BG11c medium. Afterwards, the culture performed with Arnon medium accumulated much more zeaxanthin than the other cultures.

### Effect of Light Irradiance

Table 5 reflects the effect of light irradiance on the zeaxanthin production for Anacystis nidulans. The optimum light irradiance for zeaxanthin production was found to be 1,150 μE m⁻²s⁻¹. The highest specific growth rates, zeaxanthin contents in mg l⁻¹ and zeaxanthin productions were found at irradiances from 1,610 to 690 μE m⁻²s⁻¹, productivity decreasing to half or less when irradiance was lowered from 690 to 460 μE m⁻²s⁻¹. The same trend was observed with regard to chlorophyll and zeaxanthin content in mg g⁻¹ dry weight.

TABLE 5
Effect of light irradiance

| Irradiance | | Chl (max) | Zea (max) | Zea (max) | Zea (Production) | |
|---|---|---|---|---|---|---|
| [μEm⁻²s⁻¹] | μ [h⁻¹] | [mgl⁻¹] | [mgl⁻¹] | [mgg⁻¹DW] | mgl⁻¹h⁻¹ | mgl⁻¹d⁻¹ |
| 1,610 | 0.14 | 45.7 | 13.3 | 2.9 | 0.42 | 10.1 |
| 1,150 | 0.14 | 48.7 | 13.3 | 2.8 | 0.53 | 12.7 |
| 690 | 0.13 | 56.1 | 13.2 | 2.9 | 0.47 | 11.3 |
| 460 | 0.10 | 41.2 | 7.0 | 2.2 | 0.21 | 5.0 |
| 184 | 0.06 | 31.0 | 4.4 | 1.9 | 0.13 | 3.1 |
| 92 | 0.07 | 22.3 | 2.9 | 1.5 | 0.09 | 2.2 |

### Effect of Sodium Acetate

Table 6 shows the effect of sodium acetate on growth, zeaxanthin content and zeaxanthin productivity in Anacystis nidulans. Specific growth rate was kept at about 0.1 h⁻¹ from 0 to 20 mM acetate, cells dying at 30 mM acetate. Maximum zeaxanthin in the exponential phase and at the end of the culture were kept around the same value from 0 to 15 mM acetate, decreasing at higher acetate concentrations. Optimum acetate in the medium was 15 mM, since zeaxanthin productivity was enhanced by a 27% when compared with photoautotrophic cultures.

TABLE 6
Effect of sodium acetate

| Na- | | Chl | Zea | Zea | | |
|---|---|---|---|---|---|---|
| acetate | μ | (max) | (max) | (max) | Zea (Production) | |
| [mM] | [h⁻¹] | [mgl⁻¹] | [mgl⁻¹] | [mgg⁻¹DW] | mgl⁻¹h⁻¹ | mgl⁻¹d⁻¹ |
| 0 | 0.11 | 45.5 | 11.7 | 2.9 | 0.53 | 12.7 |
| 15 | 0.12 | 37.2 | 11.5 | 3.4 | 0.67 | 16.1 |
| 20 | 0.09 | 69.8 | 9.05 | 3.3 | 0.01 | 0.34 |

What we claim is:

1. A process of obtaining zeaxanthin from blue-green algae, which process comprises:
(a) cultivating a blue-green algae selected from the group consisting of Anacystis nidulans (L-1402-1), Synechococcus sp. (PCC 7492) and Synechocystis sp. (PCC 6803) under cultivating conditions suitable for producing zeaxanthin;
(b) harvesting the cultivated algae containing zeaxanthin; and
(c) obtaining the zeaxanthin from the harvested algae containing zeaxanthin.

2. The process of claim 1 wherein the blue-green algae is Anacystis nidulans.

3. The process of claim 1 wherein the blue-green algae is cultivated at a temperature of from about 20 to about 40° C.

4. The process of claim 3 wherein the temperature is about 30° C.

5. The process of claim 1 wherein the blue-green algae is cultivated in a mixotrophic culture medium.

6. The process of claim 5 wherein the medium contains from about 0.1 to about 2 mM salts of acetic acid.

7. The process of claim 6 wherein the salt is sodium acetate.

8. The process of claim 1 wherein the blue-green algae is cultivated with a light irradiation of from about 500 to about 1,500 μ Em⁻²s⁻¹.

9. The process of claim 8 wherein the light irradiation is about 1,200 μ Em⁻²s⁻¹.

10. The process of claim 1 wherein the zeaxanthin is obtained in a yield of from about 3.0 to about 11.2 mg/liter/day.

11. The process of claim 1 which further comprises adding an antioxidant at step (b) or step (c).

12. The process of claim 11 wherein the antioxidant is alpha tocopherol.

13. A process of obtaining zeaxanthin from blue-green algae, which process comprises:
(a) cultivating a blue-green algae selected from the group consisting of Anacystis nidulans (L-1402-1), Synechococcus sp. (PCC 7492) and Synechocystis sp. (PCC 6803) under cultivating conditions suitable for producing zeaxanthin;
(b) harvesting the cultivated algae containing zeaxanthin to form a concentrated suspension of the algae;
(c) disrupting the blue-green algae of the concentrated suspension; and
(d) obtaining the zeaxanthin from the disrupted blue-green algae of the suspension.

14. The process of claim 13 wherein an antioxidant and an emulsifier are added to the concentrated suspension of step (b).

15. The process of claim 13 wherein the medium contains from about 0.1 to about 2 mM salts of acetic acid.

16. The process of claim 15 wherein the salt is sodium acetate.

17. The process of claim 13 wherein the blue-green algae is cultivated with a light irradiation of from about 500 to about 1,500 μ Em⁻²s⁻¹.

18. The process of claim 17 wherein the light irradiation is about 1,200 μ Em⁻²s⁻¹.

19. The process of claim 13 wherein the blue-green algae is Anacystis nidulans.

20. A zeaxanthin or zeaxanthin-enriched product obtained by the process of claim 1 having a zeaxanthin/lutein ratio of greater than about 10 and a chlorophyll A/zeaxanthin ratio of less than about 10.

21. A zeaxanthin or zeaxanthin-enriched product obtained by the process of claim 13 having a zeaxanthin/lutein ratio of greater than about 10 and a chlorophyll A/zeaxanthin ratio of less than about 10.
